(19) United States
(12) Patent Application Publication
(10) Pub. No.: US 2026/0026477 A1
(43) Pub. Date: Jan. 29, 2026

(54) MOUSE MODEL FOR RETINA DEGENERATION AND USES THEREOF

(71) Applicant: The Children's Medical Center Corporation, Boston, MA (US)

(72) Inventors: Ye Sun, Chestnut Hill, MA (US); Tianxi Wang, Brookline, MA (US)

(73) Assignee: The Children's Medical Center Corporation, Boston, MA (US)

(21) Appl. No.: 18/994,548

(22) PCT Filed: Jul. 14, 2023
(86) PCT No.: PCT/US2023/027803

§ 371 Date: Jan. 14, 2025

Related U.S. Application Data
(60) Provisional application No. 63/389,808, filed on Jul. 15, 2022.

(51) Int. Cl. A01K 67/0276; A61K 49/00

(52) U.S. Cl. CPC A01K 67/0276; A61K 49/0008; A01K 2207/35; A01K 2217/075; A01K 2227/105; A01K 2267/03

(57) ABSTRACT

Provided herein are irradiated mouse models which recapitulate the effects of ionizing radiation-induced microglia activation, eye injury and retinal degeneration, as well as methods for producing such mouse models. These mouse models are useful, for example, for the study of genes involved in the pathophysiology of retinal degeneration and for the development of methods for treating retinal degeneration in humans.

### RELATED APPLICATION

This application claims the benefit under 35 U.S.C. § 119(e) to U.S. Provisional Application No. 63/389,808, entitled “MOUSE MODEL FOR RETINA DEGENERATION AND USES THEREOF”, filed on Jul. 15, 2022, the entire contents of which are incorporated herein by reference.

### BACKGROUND

Tissues of the central nervous system, including the retina, are particularly sensitive to acute exposure from ionizing radiation, which can stimulate an inflammatory response and lead to irreversible cell death. The risk of acute radiation exposure is generally low for most people, however, this risk is substantially increased following a deliberate or accidental release of radioactive materials into the environment, such as, for instance, in the aftermath of a nuclear disaster. Despite the potential for ionizing radiation-induced injuries to affect millions of people per event, the genetic underpinnings that control the activation of immune cells in response to radiation exposure remain poorly understood. In addition, cranial IR as a commonly used treatment for brain tumors causes chronic inflammation and further leads to accelerated neurodegeneration in patients, particularly in children (1, 2). A deeper understanding of the factors involved in the pathophysiology of ionizing radiation-induced injuries would support the development of new approaches for treating and/or preventing these injuries.

### SUMMARY

Some aspects of the present disclosure relate to an irradiated mouse model useful for the study of ionizing radiation (IR)-induced retinal degeneration. In some aspects, the present disclosure relates to a mouse that has been exposed to a dose of ionizing radiation, wherein the mouse comprises one or more retina exhibiting microglial activation, neovascularization, and/or photoreceptor degeneration.

In some embodiments, the mouse has a wild-type (WT) genetic background. In some embodiments, the mouse has an Ai9 f/f/::LysMCre genetic background.

In some embodiments, the mouse comprises an increased number of microglial cells in the subretinal space, as compared to the number of microglial cells in the subretinal space before exposure to the dose of ionizing radiation.

In some embodiments, the mouse comprises increased growth of retinal blood vessels, as compared to the growth of retinal blood vessels before exposure to the dose of ionizing radiation.

In some embodiments, the mouse comprises an increased level of cell death in the photoreceptor layer of the retina, as compared to the level of cell death in the photoreceptor layer of the retina before exposure to the dose of ionizing radiation.

In some embodiments, the mouse comprises a retina of reduced thickness, as compared to the thickness of the retina before exposure to the dose of ionizing radiation. In some embodiments, the mouse comprises a retina comprising a photoreceptor layer of reduced thickness, as compared to the thickness of the photoreceptor layer before exposure to the dose of ionizing radiation.

In some embodiments, the dose of ionizing radiation comprises beta radiation and/or gamma irradiation. In some embodiments, the dose of ionizing radiation comprises at least 1.2 Gy of ionizing radiation. In some embodiments, the dose of ionizing radiation comprises 2.0 Gy of ionizing radiation. In some embodiments, the dose of ionizing radiation comprises 2.4 Gy of ionizing radiation.

In some embodiment, the mouse comprises an inactivated gene that is involved or suspected to be involved in microglial activation, neovascularization, and/or photoreceptor degeneration of the retina. In some embodiments, the inactivated gene is TREM2. In some embodiments, the inactivated gene is Spp1.

In some aspects, the present disclosure relates to a method for producing a mouse model described herein, such as a mouse model of ionizing radiation (IR)-induced retinal degeneration. In some aspects, the present disclosure relates to a method for producing a mouse model of IR-induced degeneration, the method comprising administering to a mouse a dose of ionizing radiation, wherein the dose of ionizing radiation is sufficient to induce microglial activation, neovascularization, and/or photoreceptor degeneration in one or more retina of the mouse.

In some embodiments, the mouse has a wild-type (WT) genetic background. In some embodiments, the mouse has an Ai9 f/f/::LysMCre genetic background.

In some embodiments, the administration results in an increased number of microglial cells in the subretinal space of the mouse, as compared to the number of microglial cells in the subretinal space of the mouse before the administration.

In some embodiments, the administration results in increased growth of retinal blood vessels, as compared to the growth of retinal blood vessels before the administration.

In some embodiments, the administration results in an increased level of cell death in the photoreceptor layer of the retina of the mouse, as compared to the level of cell death in the photoreceptor layer of the retina of the mouse before the administration.

In some embodiments, the administration results in a decrease in the retinal thickness of the mouse, as compared to the retinal thickness of the mouse before the administration. In some embodiments, the administration results in a decrease in the photoreceptor layer thickness of the mouse, as compared to the photoreceptor layer thickness of the mouse before the administration.

In some embodiments, the administration comprises beta radiation and/or gamma irradiation. In some embodiments, the administration comprises ionizing radiation from a Cesium-137 (¹³⁷Cs) radioactive source. In some embodiments, the administration comprises at least 1.2 Gy of ionizing radiation. In some embodiments, the dose of ionizing radiation comprises 2.0 Gy of ionizing radiation. In some embodiments, the administration comprises 2.4 Gy of ionizing radiation.

In some embodiments, the administration occurs within 10 days of birth of the mouse.

In some embodiments, the mouse comprises an inactivated gene that is suspected to be involved in microglial activation, neovascularization, and/or photoreceptor degeneration of the retina. In some embodiments, the method further comprises assessing whether the gene is involved in microglial activation, neovascularization, and/or photoreceptor degeneration of the retina.

Further provided herein are methods of using the mouse model described herein for developing therapeutic agents for the treatment of retinal degeneration. In some embodiments, the method comprises administering to the mouse model described herein a therapeutic agent for the treatment of retinal degeneration. In some embodiments, the method further comprises assessing the effectiveness of the drug in treating retinal degeneration.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows a schematic of the experimental design for producing an irradiation-induced mouse model of retinal degeneration. A mouse (younger than at postnatal day 10) is irradiated without use of a head shield using a Cs-137 irradiator with a dose of 2.4 Gy. The mouse eye is collected at day 5 (D5), day 11 (D11), day 17 (D17), and day 60 (D60) post irradiation (IR) to examine microglia accumulation/activation and photoreceptor degeneration. Optical coherence tomography (OCT) is used to examine the photoreceptor degeneration in live mice.

FIGS. 2A-2E show microglial activation and photoreceptor degeneration in irradiated mice over time. FIG. 2A shows microglial activation in an irradiated reporter mouse strain. Ai9 f/f::LysMCre reporter mice were irradiated at D1 and the mouse eye was collected at day 5 (D5) post-irradiation. The retina was dissected and imaged. Many microglia appeared in the subretinal space, between the retina and retinal pigment epithelium (RPE). FIG. 2B shows cell death occurring in the photoreceptor layer of irradiated mice. Wild type mice (WT) were irradiated at D1 and the eye was collected at day 11 (D11) post-irradiation. TUNEL staining was used to examine cell death occurring in the retina post-irradiation. TUNEL positive cells are observed in the photoreceptor layer. On the surface of retina (facing the RPE side), circular regions are observed (left; encircled regions), as are cells in those regions (right; arrows) as indicated by DAPI, which could further indicate the presence of microglia. FIG. 2C shows positive identification of microglia. WT mice were irradiated and eyes were collected at day 17 (D17) post-irradiation for immunostaining with microglial marker IBA1 to further confirm the presence of microglia. FIG. 2D shows the presence of GFP+ bone marrow derived cells in the “hole” regions observed in FIG. 2C, which partially colocalize with microglia marker IBA1. FIG. 2E shows optical coherence tomography (OCT) conducted on live retina following irradiation. WT mice were irradiated and OCT was performed at day 60 (D60) post-irradiation to examine the retinal layer thickness to further confirm photoreceptor degeneration in irradiated mice. After irradiation, the thickness of the retinal layer was reduced.

FIG. 3 shows an IR-induced retinal degeneration mouse model. Mice were irradiated at the dose of 2.4 G at postnatal day 8 and the retinal layer thickness was examined using OCT at postnatal day 30.

FIG. 4 shows an IR-induced retinal degeneration mouse model. Mice were irradiated at the dose of 2.4 G at postnatal day 8 and the retinal layer thickness was examined using OCT at postnatal day 60.

FIG. 5 shows a model for retinal degeneration in irradiated mice. Irradiation-induced microglia accumulate in the subretinal space between the retina and RPE and further lead to photoreceptor degeneration.

FIGS. 6A and 6B show induction of TREM2 in a mouse model of ionizing radiation-induced retinal degeneration (IR-RD) and in a laser induced neovascularization mouse model. FIG. 6A shows representative retinal mounts from wild-type (WT), rd10, Rho^{P23H/+}, and IR-RD mice. Retinal mounts were immunostained for TREM2 and a microglial marker, IBA1. FIG. 6B shows choroidal flat mounts from mice with laser induced neovascularization (NV). Choroidal flat mounts were immunostained for TREM2 and IBA1, and DAPI-stained to indicate nuclei. Enlarged images of boxed regions “A” and “B” are shown at right.

FIG. 7 shows microglia in an IR-induced RD mouse model. Mice were irradiated at the dose of 2.4 G at postnatal day 8. The eyes were collected to examine the expression of Trem2 and Spp1 were examined at P60. Trem2 and Spp1 double expressing microglia were found in IR-RD mouse model.

FIG. 8 shows microglia in genetic RD mouse models. The eyes from 10-week-old P23H and 12-week-old Rd10 mice were collected to examine the expression of Trem2 and Spp1 were examined. Trem2+ Spp1+ microglia were found in these RD mouse models.

FIG. 9A-9B show IR-RD mouse model were generated using S129 mice. Representative mouse retinal flat mount images from the mice were irradiated with 2.4 G at indicated time points.

FIG. 10 shows microglia that were examined in IR-RD mouse model generated using S129 mice. Representative mouse retinal flat mount images from the mice were irradiated with 2.4 G at indicated time points.

FIG. 11 show IR-induced retinal microglia activation. Microglia were examined in IR-RD mouse model generated using S129 mice. Representative mouse retinal flat mount images from the mice were irradiated with 2.4 G at postnatal day 2.

FIG. 12 shows IR-induced retinal microglia activation. Microglia were examined in IR-RD mouse model generated using C57BL6 mice. Representative mouse retinal flat mount images from the mice were irradiated with 2.4 G at postnatal day 2, 3, 4, 5, 6, or 7 and the eyes were collected and examined at postnatal day 15.

FIG. 13A-13C show microglia that were examined in IR-RD mouse model generated using C57BL6 mice. Representative mouse retinal flat mount images from the mice were irradiated with 2.4 G at postnatal day 5 (P5).

FIG. 14A-14C show microglia that were examined in IR-RD mouse model generated using myeloid Socs3 knockout mice (Socs3 ff:LysMCre) and Socs3 floxed control (Socs3 ff) mice. Representative mouse retinal flat mount images from the mice were irradiated with 2.4 G at postnatal day 2 (P2).

FIG. 15 shows microglia that were examined in IR-RD mouse model generated using Trem2 knockout mice (Trem2 KO). Representative mouse retinal flat mount images from Trem2 KO mice were irradiated with 2.4 G at postnatal day 1 (P1) and retinal flat mounts at P6 were stained with IB4 and IBA1.

FIG. 16 shows microglia that were examined in IR-RD mouse model generated using Trem2 knockout mice (Trem2 KO). Representative mouse retinal flat mount images from Trem2 KO mice were irradiated with 2.4 G at postnatal day 1 (P1) and retinal flat mounts at P17 were stained with IB4 and IBA1.

FIG. 17 shows microglia that were examined in IR-RD mouse model generated using Spp1 knockout mice (Spp1 KO). Representative mouse retinal flat mount images from Spp1 KO mice were irradiated at postnatal day 1 (P1) and retinal flat mounts at P6 were stained with IBA1.

FIGS. 18A-18B show microglia that were examined in IR-RD mouse model generated using Spp1 knockout mice (Spp1 KO) and S129 mice. Representative mouse retinal flat mount images from Spp1 KO mice and S129 were irradiated at postnatal day 1 (P1) and retinal flat mounts at P13 were stained with IBA1.

FIGS. 19A-19B show microglia that were examined in IR-RD mouse model generated using Ai9-LysMCre reporter mice (FIG. 19A). Representative mouse retinal flat mount images from Ai9 lysmcre mice irradiated at postnatal day 1 (P1) and retinal flat mounts at P5 were examined for microglia labeled with tdTomato and the cell number of microglia was quantified (FIG. 19B).

### DETAILED DESCRIPTION OF CERTAIN EMBODIMENTS

Aspects of the present disclosure are based on the development of a mouse model that recapitulates irradiation-induced retinal diseases, such as, for example, ionizing radiation (IR)-induced retinal degeneration (RD). Due to the ongoing global threat of radiation exposure from warfare and accidental release, the neurobiological impact of high and low doses of IR and responses by biological systems to IR exposure needs to be revaluated. High doses of IR have been previously reported to cause neural injury, which may lead to neurodegenerative diseases. Acute exposure of IR has manifold effects on the central nervous system and induces inflammatory responses via microglia activation. The retina is an extension of the brain, and high doses of IR can induce RD. However, the mechanisms underlying the IR-induced RD is largely unknown. To a large extent, this lack of mechanistic understanding reflects the limitations of current model systems for pursuing such studies. The mouse model described herein is useful, for instance, for facilitating these studies with the goal of developing new techniques for treating and preventing IR-induced RD.

### Mouse Models

As described in the Examples provided herein, a previously unreported mouse model has been developed which is exposed to a dose of IR sufficient to induce several traits of IR-induced RD, including, but not limited to, microglial activation, neovascularization, and photoreceptor degeneration. Without wishing to be bound by theory, the effects of IR observed in this mouse model recapitulate the effects of IR observed during IR-induced RD in other mammalian species, including humans. For this reason, this mouse model is useful for the study of genes involved in IR-induced RD in humans, and could be used in inform new treatments for IR-induced RD.

In some aspects, the present disclosure provides an irradiated mouse which has been exposed to a dose of IR. In some embodiments, the irradiated mouse comprises one (1) or more (e.g., two (2)) retina exhibiting microglial activation, neovascularization, and/or photoreceptor degeneration. In some embodiments, the irradiated mouse has a wild-type (WT) genetic background. In some embodiments, a WT mouse is a mouse that does not express any transgene (e.g., a mouse transgene or a human transgene). In some embodiments, the irradiated mouse expresses one or more transgenes, such as one or more genetic reporters for microglial activation. In some embodiments, the mouse has an Ai9 f/f/::LysMCre genetic background, or expresses another genetic reporter known to those of ordinary skill in the art. Examples of other genetic reporters, include, but are not limited to, Ai9f/f :: Cx3cr1 Cre, Ai9f/f:: Tmem119 Cre, Ai9f/f:: P2ry12 Cre, Cx3cr1-GFP, and mTmG::Cx3cr1Cre. In some embodiments, the irradiated mouse has a mutant genetic background. In some embodiments, a mutant genetic background is caused by at least 1 (e.g., at least 1, at least 2, at least 3, at least 4, at least 5) mutations. In some embodiments, the mutation(s) in the mouse genome results in an inactivated gene. In some embodiments, the inactivated gene is involved or suspected to be involved in microglial activation, neovascularization, and/or photoreceptor degeneration of the retina. In some embodiments, the inactivated gene is TREM2 and/or Spp1. In some embodiments, the inactivated gene is TREM2 or Spp1. In some embodiments, the inactivated gene is TREM2 and Spp1. In some embodiments, the inactivated gene is TREM2. In some embodiments, the inactivated gene is TREM2 or Spp1.

In some embodiments, the irradiated mouse comprises an increased number of microglial cells in the retina and/or in the subretinal space. In some embodiments, the number of microglial cells in the retina and/or in the subretinal space is increased as compared to the number of microglial cells in the retina and/or in the subretinal space of the mouse before exposure to the dose of IR. In some embodiments, the number of microglial cells in the retina and/or in the subretinal space is increased as compared to the number of microglial cells in the retina and/or in the subretinal space of an age- and sex-matched control mouse that is not exposed to the dose of IR. In some embodiments, the number of microglial cells in the retina and/or in the subretinal space of the mouse is increased by up to 10%, up to 20%, up to 30%, up to 40%, up to 50%, up to 60%, up to 70%, up to 80%, up to 90%, up to 2-fold, up to 3-fold, up to 4-fold, up to 5-fold, up to 6-fold, up to 7-fold, up to 8-fold, up to 9-fold, or up to 10-fold or more.

In some embodiments, the irradiated mouse comprises increased growth of retinal blood vessels (retinal neovascularization). In some embodiments, the growth of retinal blood vessels is increased as compared to the growth of retinal blood vessels in the mouse before exposure to the dose of IR. In some embodiments, the growth of retinal blood vessels is increased as compared to the growth of retinal blood vessels of an age- and sex-matched control mouse that is not exposed to the dose of IR. In some embodiments, the growth of retinal blood vessels is increased by up to 10%, up to 20%, up to 30%, up to 40%, up to 50%, up to 60%, up to 70%, up to 80%, up to 90%, up to 2-fold, up to 3-fold, up to 4-fold, up to 5-fold, up to 6-fold, up to 7-fold, up to 8-fold, up to 9-fold, or up to 10-fold or more.

In some embodiments, the irradiated mouse comprises an increased level of cell death (e.g., photoreceptor cell death) in the photoreceptor layer of the retina. In some embodiments, the level of cell death (e.g., photoreceptor cell death) in the photoreceptor layer of the retina is increased as compared to the level of cell death (e.g., photoreceptor cell death) in the photoreceptor layer of the retina before exposure to the dose of IR. In some embodiments, the level of cell death (e.g., photoreceptor cell death) in the photoreceptor layer of the retina is increased as compared to the level of cell death (e.g., photoreceptor cell death) in the photoreceptor layer of the retina of an age- and sex-matched control mouse that is not exposed to the dose of IR. In some embodiments, the level of cell death (e.g., photoreceptor cell death) in the photoreceptor layer of the retina is increased by up to 10%, up to 20%, up to 30%, up to 40%, up to 50%, up to 60%, up to 70%, up to 80%, up to 90%, up to 2-fold, up to 3-fold, up to 4-fold, up to 5-fold, up to 6-fold, up to 7-fold, up to 8-fold, up to 9-fold, or up to 10-fold or more.

In some embodiments, the irradiated mouse comprises a retina of reduced thickness. In some embodiments, the thickness of the retina is reduced as compared to the thickness of the retina of the mouse before exposure to the dose of IR. In some embodiments, the thickness of the retina is reduced as compared to the thickness of the retina of an age- and sex-matched control mouse that is not exposed to the dose of IR. In some embodiments, the thickness of the retina of the mouse is reduced by up to 10%, up to 20%, up to 30%, up to 40%, up to 50%, up to 60%, up to 70%, up to 80%, up to 90%, up to 95%, up to 99%, or up to 100%.

In some embodiments, the irradiated mouse comprises a retina comprising a photoreceptor layer of reduced thickness. In some embodiments, the thickness of the photoreceptor layer of the retina is reduced as compared to the thickness of the photoreceptor layer of the retina of the mouse before exposure to the dose of IR. In some embodiments, the thickness of the photoreceptor layer of the retina is reduced as compared to the thickness of the photoreceptor layer of the retina of an age- and sex-matched control mouse that is not exposed to the dose of IR. In some embodiments, the thickness of the photoreceptor layer of the retina of the mouse is reduced by up to 10%, up to 20%, up to 30%, up to 40%, up to 50%, up to 60%, up to 70%, up to 80%, up to 90%, up to 95%, up to 99%, or up to 100%.

In some embodiments, the dose of IR comprises exposure to one or more radioactive isotopes. In some embodiments, the dose of IR comprises alpha radiation (alpha decay). In some embodiments, the dose of IR comprises beta radiation (beta decay). In some embodiments, the dose of IR comprises gamma radiation (gamma decay). In some embodiments, the dose of IR comprises beta radiation and gamma radiation. In some embodiments, the dose of IR comprises exposure to Cesium-137 (¹³⁷Cs), or another suitable radioactive source (e.g., a radioactive source of beta radiation and gamma radiation) known to those of ordinary skill in the art. In some embodiments, the dose of IR comprises at least 0.5 gray of IR (1 Gy=J/kg), e.g., at least 0.5 Gy, at least 0.6 Gy, at least 0.7 Gy, at least 0.8 Gy, at least 0.9 Gy, at least 1.0 Gy, at least 1.1 Gy, at least 1.2 Gy, at least 1.3 Gy, at least 1.4 Gy, at least 1.5 Gy, at least 1.6 Gy, at least 1.7 Gy, at least 1.7 Gy, at least 1.8 Gy, at least 2.0 Gy, at least 2.1 Gy, at least 2.2 Gy, at least 2.3 Gy, at least 2.5 Gy, at least 2.6 Gy, at least 2.7 Gy, at least 2.8 Gy, at least 2.9 Gy, or at least 3.0 Gy. In some embodiments, the dose of IR comprises at least 2.0 Gy or IR. In some embodiments, the dose of IR comprises 2.4 Gy of IR.

In some embodiments, the irradiated mouse has been postnatally exposed to the dose of IR. In some embodiments, the irradiated mouse has been exposed to the dose of IR within 10 days of birth, e.g., within 1 day, 2 days, 3 days, 4 days, 5 days, 6 days, 7 days, 8 days, 9 days, or 10 days of birth. In some embodiments, the irradiated mouse has been exposed to the dose of IR after 10 days of birth. In some embodiments, the irradiated mouse has been exposed to the dose of IR 11 days, 12 days, 13 days, 2 weeks, 3 weeks, 1 month, 2 months, 3 months, 4 months, 5 months, 6 months, 7 months, 8 months, 9 months, 10 months, 11 months, or 1 year after birth. In some embodiments, the irradiated mouse is exposed to the dose of IR more than 1 year after birth.

### Methods of Producing Mouse Models

Further aspects of the present disclosure relate to methods for producing a mouse model described herein. In one aspect, the present disclosure provides a method for producing a mouse model of IR-induced RD, the method comprising administering to a mouse a dose of IR. In some embodiments, the dose of IR is sufficient to induce microglial activation, neovascularization, and/or photoreceptor degeneration in one (1) or more (e.g., two (2)) retina of the mouse. In some embodiments, the mouse has a wild-type (WT) genetic background. In some embodiments, the mouse has an Ai9 f/f/::LysMCre genetic background, or expresses another transgene (e.g., a transgene encoding a genetic reporter) known to those of ordinary skill in the art.

In some embodiments, the administration results in an increased number of microglial cells in the retina and/or in the subretinal space of the mouse. In some embodiments, the number of microglial cells in the retina and/or in the subretinal space is increased as compared to the number of microglial cells in the retina and/or in the subretinal space of the mouse before the administration. In some embodiments, the number of microglial cells in the retina and/or in the subretinal space is increased as compared to the number of microglial cells in the retina and/or in the subretinal space of an age- and sex-matched control mouse that is not administered a dose of IR. In some embodiments, the number of microglial cells in the retina and/or in the subretinal space of the mouse is increased by up to 10%, up to 20%, up to 30%, up to 40%, up to 50%, up to 60%, up to 70%, up to 80%, up to 90%, up to 2-fold, up to 3-fold, up to 4-fold, up to 5-fold, up to 6-fold, up to 7-fold, up to 8-fold, up to 9-fold, or up to 10-fold or more as a result of the administration.

In some embodiments, the administration results in increased growth of retinal blood vessels (retinal neovascularization) in the mouse. In some embodiments, the growth of retinal blood vessels in the mouse is increased as compared to the growth of retinal blood vessels in the mouse before the administration. In some embodiments, the growth of retinal blood vessels in the mouse is increased as compared to the growth of retinal blood vessels in an age- and sex-matched control mouse that is not administered a dose of IR. In some embodiments, the growth of retinal blood vessels in the mouse is increased by up to 10%, up to 20%, up to 30%, up to 40%, up to 50%, up to 60%, up to 70%, up to 80%, up to 90%, up to 2-fold, up to 3-fold, up to 4-fold, up to 5-fold, up to 6-fold, up to 7-fold, up to 8-fold, up to 9-fold, or up to 10-fold or more as a result of the administration.

In some embodiments, the administration results in an increased level of cell death (e.g., photoreceptor cell death) in the photoreceptor layer of the retina of the mouse. In some embodiments, the level of cell death (e.g., photoreceptor cell death) in the photoreceptor layer of the retina of the mouse is increased as compared to the level of cell death (e.g., photoreceptor cell death) in the photoreceptor layer of the retina of the mouse before the administration. In some embodiments, the level of cell death (e.g., photoreceptor cell death) in the photoreceptor layer of the retina of the mouse is increased as compared to the level of cell death (e.g., photoreceptor cell death) in the photoreceptor layer of the retina of an age- and sex-matched control mouse that is not administered a dose of IR. In some embodiments, the level of cell death (e.g., photoreceptor cell death) in the photoreceptor layer of the retina of the mouse is increased by up to 10%, up to 20%, up to 30%, up to 40%, up to 50%, up to 60%, up to 70%, up to 80%, up to 90%, up to 2-fold, up to 3-fold, up to 4-fold, up to 5-fold, up to 6-fold, up to 7-fold, up to 8-fold, up to 9-fold, or up to 10-fold or more as a result of the administration.

In some embodiments, the administration results in a retina of reduced thickness in the mouse. In some embodiments, the thickness of the retina of the mouse is reduced as compared to the thickness of the retina of the mouse before the administration. In some embodiments, the thickness of the retina of the mouse is reduced as compared to the thickness of the retina of an age- and sex-matched control mouse that is not administered a dose of IR. In some embodiments, the thickness of the retina of the mouse is reduced by up to 10%, up to 20%, up to 30%, up to 40%, up to 50%, up to 60%, up to 70%, up to 80%, up to 90%, up to 95%, up to 99%, or up to 100% as a result of the administration.

In some embodiments, the administration results a retina of the mouse comprising a photoreceptor layer of reduced thickness. In some embodiments, the thickness of the photoreceptor layer of the retina of the mouse is reduced as compared to the thickness of the photoreceptor layer of the retina of the mouse before the administration. In some embodiments, the thickness of the photoreceptor layer of the retina of the mouse is reduced as compared to the thickness of the photoreceptor layer of the retina of an age- and sex-matched control mouse that is not administered the IR. In some embodiments, the thickness of the photoreceptor layer of the retina of the mouse is reduced by up to 10%, up to 20%, up to 30%, up to 40%, up to 50%, up to 60%, up to 70%, up to 80%, up to 90%, up to 95%, up to 99%, or up to 100% as a result of the administration.

In some embodiments, the administration comprises one or more radioactive isotopes. In some embodiments, the administration comprises alpha radiation (alpha decay). In some embodiments, the administration comprises beta radiation (beta decay). In some embodiments, the administration comprises gamma radiation (gamma decay). In some embodiments, the administration comprises beta radiation and gamma radiation. In some embodiments, the administration comprises exposure to Cesium-137 (¹³⁷Cs), or another suitable radioactive source (e.g., a radioactive source of beta radiation and gamma radiation) known to those of ordinary skill in the art. In some embodiments, the administration comprises at least 0.5 gray of IR (1 Gy=J/kg), e.g., at least 0.5 Gy, at least 0.6 Gy, at least 0.7 Gy, at least 0.8 Gy, at least 0.9 Gy, at least 1.0 Gy, at least 1.1 Gy, at least 1.2 Gy, at least 1.3 Gy, at least 1.4 Gy, at least 1.5 Gy, at least 1.6 Gy, at least 1.7 Gy, at least 1.7 Gy, at least 1.8 Gy, at least 2.0 Gy, at least 2.1 Gy, at least 2.2 Gy, at least 2.3 Gy, at least 2.5 Gy, at least 2.6 Gy, at least 2.7 Gy, at least 2.8 Gy, at least 2.9 Gy, or at least 3.0 Gy. In some embodiments, the administration comprises at least 2.0 Gy or IR. In some embodiments, the administration comprises 2.4 Gy of IR.

In some embodiments, the mouse is postnatally administered the dose of IR. In some embodiments, the mouse is administered the dose of IR within 10 days of birth, e.g., within 1 day, 2 days, 3 days, 4 days, 5 days, 6 days, 7 days, 8 days, 9 days, or 10 days of birth. In some embodiments, the mouse is administered the dose of IR after 10 days of birth. In some embodiments, the mouse is administered the dose of IR 11 days, 12 days, 13 days, 2 weeks, 3 weeks, 1 month, 2 months, 3 months, 4 months, 5 months, 6 months, 7 months, 8 months, 9 months, 10 months, 11 months, or 1 year after birth. In some embodiments, the mouse is administered the dose of IR more than 1 year after birth.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1: Development of a Murine Model for Ionizing Radiation (IR)-Induced Retinal Degeneration (RD)

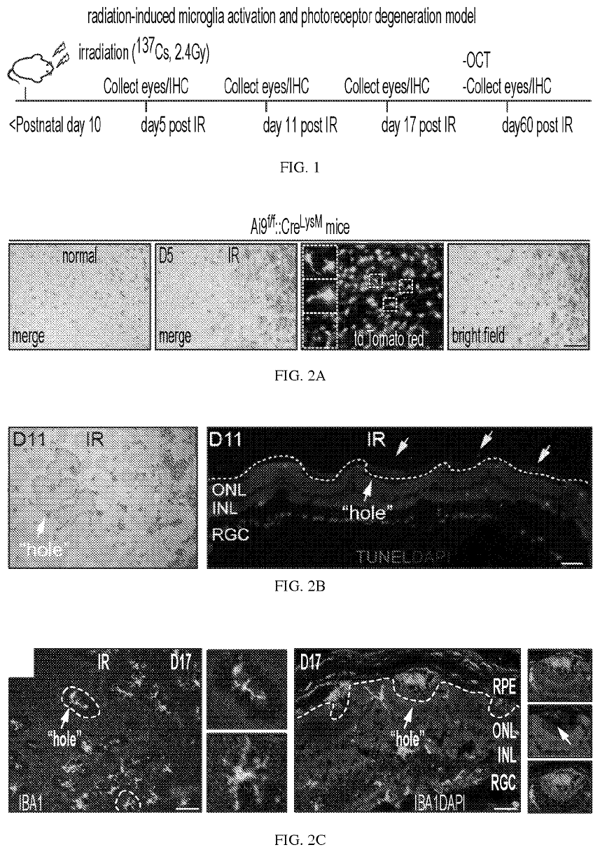

A murine model for ionizing radiation (IR)-induced retinal degeneration (RD) was developed by irradiating mice younger than postnatal day 10 with a Cs-137 irradiator without use of a head shield. Mice were weighed and irradiated with a 2.4 Gy dose of IR. After irradiation, microglial activation and photoreceptor degeneration were examined by sacrificing mice at different time points and excising retina for further analysis. Select mice were also examined by optical coherence tomography (OCT) to assess photoreceptor degeneration in live mice (FIG. 1).

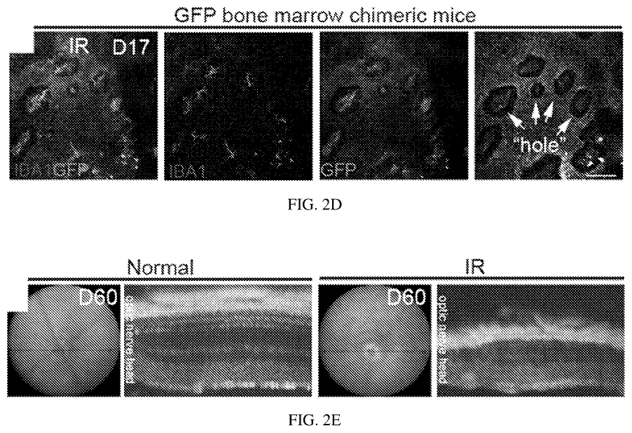

First, mice expressing a Ai9 f/f:: LysMCre reporter were examined for the presence of myeloid lineage cells in the retina or subretinal space between the retina and retinal pigment epithelium (RPE) (FIG. 2A). At 5 days post-irradiation, Ai9 f/f:: LysMCre mice were observed to have many myeloid cells present in the subretinal space, which is indicative of an increase in microglial activation and accumulation. Subsequently, wild-type (WT) mice were examined for occurring cell death in the retina, a hallmark of retinal degeneration (FIG. 2B). TUNEL positive cells were observed to appear in the photoreceptor layer within 11 days after irradiation, while additional structures were observed that could potentially represent microglia, supporting the results observed in FIG. 2A. To confirm the presence of microglia, in the subretinal space, retinas from WT mice 17 days post-irradiation were assayed for the microglial-specific marker IBA1 (FIG. 2C). To investigate the cell origin of these cells, GFP bone marrow chimeric mice were dosed with IR and the presence of microglia was examined (FIG. 2D). GFP+ bone marrow derived cells were present in “hole” shaped regions of the retina and only partially colocalized with microglia marker IBA1, suggesting that these cells are primarily local microglia and only partially originate from peripheral bone marrow derived cells. Finally, retinas of live mice were examined by OCT at 60 days post-irradiation to confirm reduced photoreceptor layer thickness (FIG. 2E). As expected, even at 60 days post-irradiation, mice exhibited photoreceptor layer loss indicative of IR-induced RD. These data support a model for IR-induced RD in mice, in which initial accumulation of microglia in the subretinal space is followed by cell death and thinning of the photoreceptor layer, leading to loss of visual acuity.

### Example 2: Utilization of a Murine Model for Ionizing Radiation (IR)-Induced Retinal Degeneration (RD) for the Study of Factors Involved in IR-RD Pathophysiology

As described in Example 1, a mouse model of IR-induced microglia activation and RD has been developed. Through the use of this model, the role of microglia in IR-induced RD via a microglial gene, TREM2 (Triggering receptor expressed on myeloid cells 2) may be explored. Using TREM2 knockout (KO) mice in a laser-induced choroidal neovascularization (CNV) mouse model, the role of microglial TREM2 in controlling laser-induced CNV that could lead to vision loss may be further examined. These studies will illustrate the influence of IR in visual dysfunction caused by IR exposure, and uncover the novel molecular mechanism in IR-induced RD and laser-induced CNV, which may lead to new approaches for preventing IR-induced RD and laser-induced CNV after IR or laser exposure, respectively. These studies will further demonstrate the mechanisms of injury for visual system trauma secondary to directed energy such as IR and laser, as well as demonstrate the validity of the IR-RD mouse model.

Microglia may be modulated via TREM2 to reduce IR-induced RD and laser-induced CNV. Neuroinflammation triggered by dead cells, oxidative stress, and oxidized lipoproteins caused by IR or laser exposure contributes to the initiation of RD and CNV, and is a key characteristic of neuroinflammation is activation of microglia⁽³⁾. Preliminary data shown in Example 1 indicate that IR can induce microglia activation in subretinal space that contributes to RD development (FIG. 1 and FIGS. 2A-2E). Previous studies have shown that microglia contribute to NV formation⁽⁴⁾, and that the expression of TREM2 was altered in microglia under various pathological conditions⁽⁵⁻⁸⁾. Taken together, it is plausible that targeting microglia via TREM2 can reduce IR-induced RD and laser-induced NV by controlling microglia function. It remails unknown however how TREM2 controls RD pathophysiology and visual function.

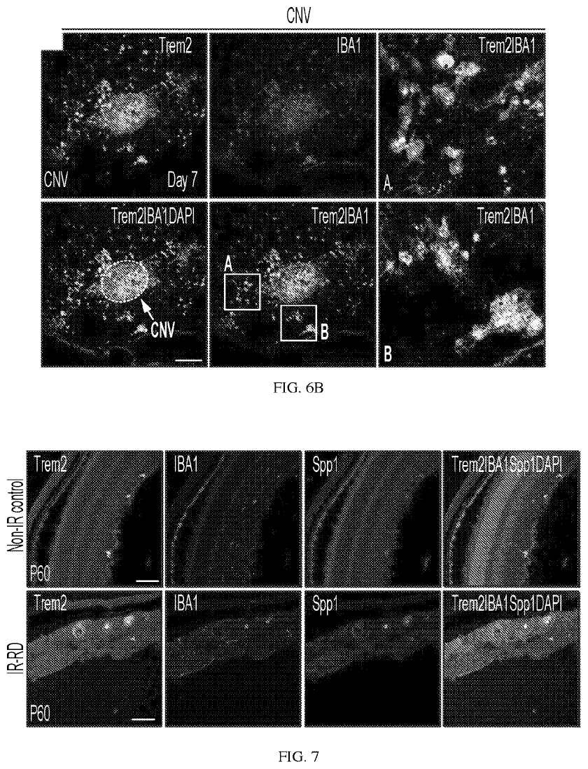

To investigate the effect of TREM2 using the IR-RD mouse model, retinas from RD mouse models (rd10 and Rho^{P23H/+}mice; positive control), control untreated wild-type (WT) mice, and WT mice dosed with IR were immunostained for TREM2 and the active microglia marker IBA1. An increase in active microglia was observed in all RD models as compared with the WT control, and TREM2 was expressed in active microglia (FIG. 6A), suggesting a critical role of TREM2 in RD, and specifically in IR-RD. For comparison, choroid with laser-induced CNV were also examined from wild type mice and immunostained with TREM2 and IBA1. TREM2⁺ IBA1⁺ microglia were observed in the neovascularization area, further indicating the involvement of TREM2⁺ microglia in CNV development (FIG. 6B).

To further evaluate the role of TREM2, Trem2^{KO} mice and littermate Trem2^{WT} control mice may be subjected to IR at different doses and the thicknesses of retinal layers assessed at postnatal day (P) 60, P90, and P120 via Micro IV Retinal Image Guided OCT2^{(9, 10)} (Phoenix). InSight software (Phoenix) can be used to analyze the retinal thicknesses^{(9, 10)}. Visual function of mice may be assessed by eletroretinogram⁽¹¹⁾ at P60, P90, and P120. The saturating sensitivity of the rod photo-response may be estimated by fitting a model of the biochemical processes involved in the activation of phototransduction to electroretinogram a-waves⁽¹²⁾. Similar studies may be performed in laser-induced CNV mice that are 6-8-week-old littermates. Choroid may be dissected at day (d)3, d5 and d7 after laser induction. CNV and leakage from neovascularization can be analyzed using known methods⁽¹³⁾.

Finally, the IR-induced RD model and laser-induced CNV model may be used to further evaluate microglial migration during IR-RD and laser-induced CNV, respectively. For example, Trem2^{KO} and littermate Trem2^{WT} control mice may be subjected to IR-induced RD or laser-induced NV model. In the IR-induced RD model, eyes may be collected at day 7, 15, 30, and 60 after IR. In laser-induced NV model, eyes may be collected at day 1, 3, 5, and 7 after laser induction. Retina, choroid, and RPE can be dissected for immunohistochemical staining on whole mount or cross sections with known microglia markers, including anti-P2ry12, anti-CD11b, and anti-IBA1. The z-stack images of whole mounts can be captured using confocal microscopy (e.g., LSM800 with Airyscan, Zeiss) for 3D reconstruction in order to observe the microglia morphology and quantity at different time points during the progression of RD and CNV. For instance, six confocal images may be taken per retina and 3 mice per group may be included for quantification.

As described above, the studies are useful for uncovering the molecular mechanisms underlying microglial activation during IR-induced RD and laser-induced CNV, and for the eventual identification of therapeutic targets for treating or preventing IR-induced RD and laser-induced CNV after IR or laser exposure. These studies are necessary to provide broad biological insights on IR and laser exposure in visual system trauma research and provide new targets for developing therapeutic strategies for IR-induced RD and laser-induced NV. Furthermore, these studies will further demonstrate the utility of a new animal model for microglia activation caused by IR and lasers. The concepts and approaches described here may be applicable to other fields as well, such as microglial activation induced-neurodegeneration and angiogenesis.

### Example 3: Trem2-Expressing Microglia Were Examined in IR-RD Mouse Model Compared to Other RD Mouse Models

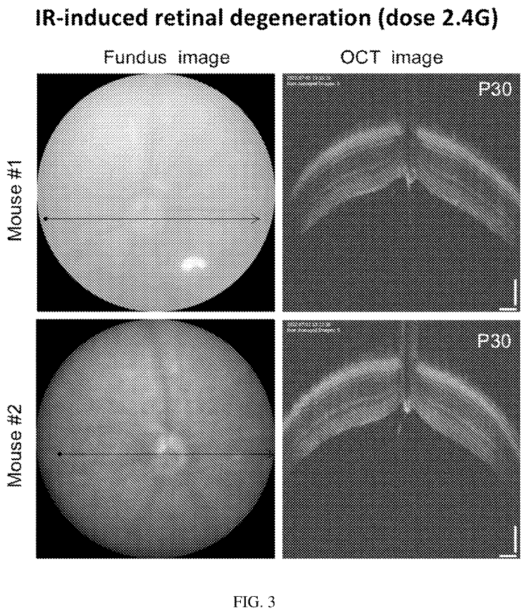

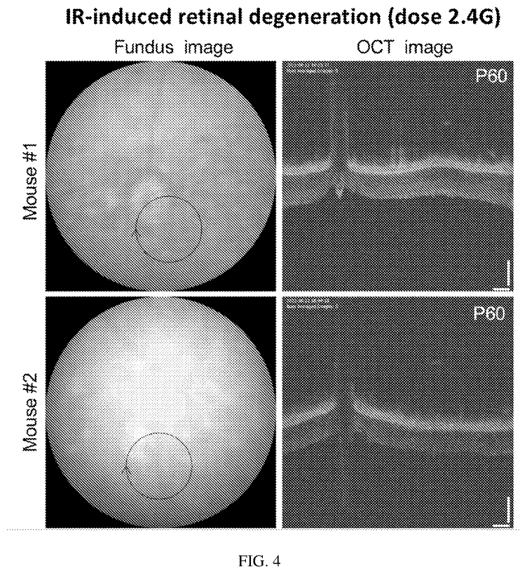

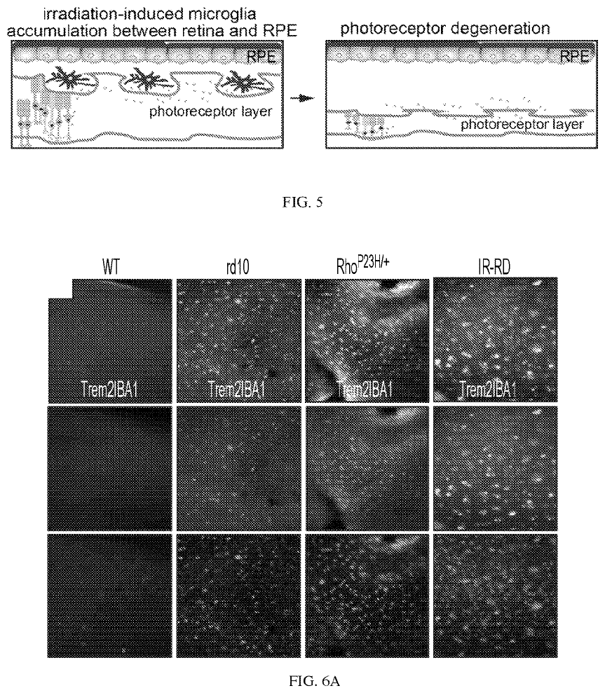

To investigate how microglia control the development of RD, a mouse model of IR-induced microglia activation was developed and examined the RD at different time points (postnatal day 30 in FIG. 3 and postnatal 60 at FIG. 4. Retinal degeneration was clearly observed at postnatal 60 (P60). All the outer nuclear layer had disappeared. This data suggested that IR-RD was successfully generated.

Comprehensive single-cell sequencing analysis of Alzheimer's Disease (AD), amyotrophic lateral sclerosis, and aging identified a new subset of microglia, named as disease associated microglia (DAM), conserved in mice and humans (3,4). DAM is associated with expression of genes are linked to AD and other neurodegenerative diseases including triggering receptor expressed on myeloid cells 2 (Trem2), a receptor required for DAM activation (3,4). Deficiency of Trem2 demonstrated that it is essential for microglia to respond to neurodegeneration cue in mouse model of neurodegenerative disease (5). Therefore, DAM is a universal immune sensor of neurodegeneration (4). However, whether DAM occur in the IR-induced RD and what is the role of DAM in IR-induced RD are completely unknown. The data showed that Trem2 was induced in IR-induced RD model (IR-RD) after IR exposure and genetically modified RD mouse models (FIG. 3). There were more active microglia labeled by IBA1 in known RD mouse models (rd10(6) and RhoP23H/+mice (7)) and IR-RD model compared with wild type control, and Trem2 was expressed in active microglia (FIG. 3). Therefore, the data provided the evidence that Trem2-expressing DAM may appear in the IR-induced RD mouse model as well as other RD mouse models (FIG. 3), suggesting that IR exposure could induce this new subset of Trem2-expressing microglia that are similar with the DAM in the brains with AD.

Trem2-expressing microglia in IR-RD mouse model showed similar patterns with microglia in other RD mouse models (rd10, RhoP23H mice) that were widely used in RD research. Targeting DAM via Trem2 may treat IR-induced RD by controlling microglia activation.

### Example 4: The Marker Genes of Microglia Were Examined in IR-Induced Microglia in IR-RD Mouse Model

The scRNA-seq data analysis showed that the relative abundance of several clusters of microglia were increased in myeloid Socs3 cKO mice with oxygen-induced retinopathy (OIR) and these microglia highly expressed secreted phosphoprotein 1 (SPP1). SPP1 is a cytokine that upregulates expression of interferon-gamma and interleukin-12 (8), and is expressed by microglia and macrophage subsets, T cells, and other immune cells to modulate various macrophage functions, including phagocytosis. In choroidal neovascular membranes surgically extracted from human age-related macular degeneration patients, SPP1 expression is increased and numerous SPP1 positive microglia are observed (9). The data showed that Spp1 was highly induced in the neovascularization areas in OIR mice and laser-induced choroidal neovascularization mice, particularly in the mice lacking myeloid SOCS3, and human patients with neovascular age-related macular degeneration. The knockout of Spp1 suppressed myeloid SOCS3 deficiency-induced neovascularization. These findings suggest that Spp1+ microglia contribute to the development of ocular neovascularization. Therefore, the Spp1 expression was examined in IR-RD mouse model and found that Spp1+ microglia were found in this mouse model and colocalized with Trem2 expression (FIG. 7). These findings suggest that IR induced microglia mouse model can be used to study Trem2+ microglia and Spp1+ microglia.

### Example 5: The Marker Genes of Microglia Were Examined in Genetic Modified RD Mouse Model

The marker gene expression of microglia in other retinal degeneration mouse models including rd10 mice and RhoP23H mutant (P23H) mice was also investigated. Both Spp1 and Trem2 were expressed in microglia in these mouse models during the progression of retinal degeneration (FIG. 8). These findings suggest the Spp1 and Trem2 double positive microglia found in IR-RD mouse model were also found in other genetic modified RD mouse models.

### Example 6: IR-RD Mouse Model was Examined in Mice With Different Genetic Background—S129 Mice

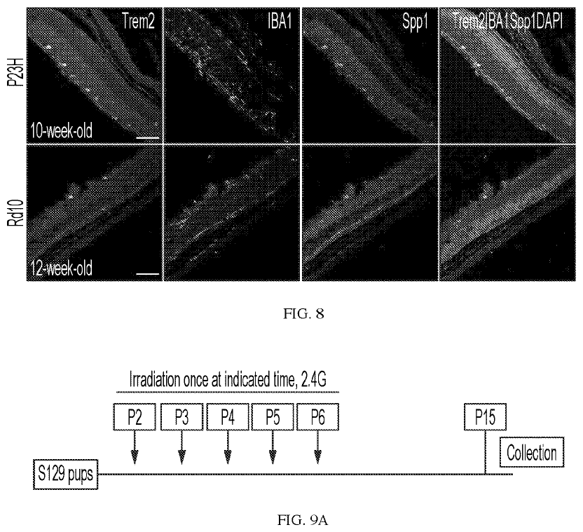

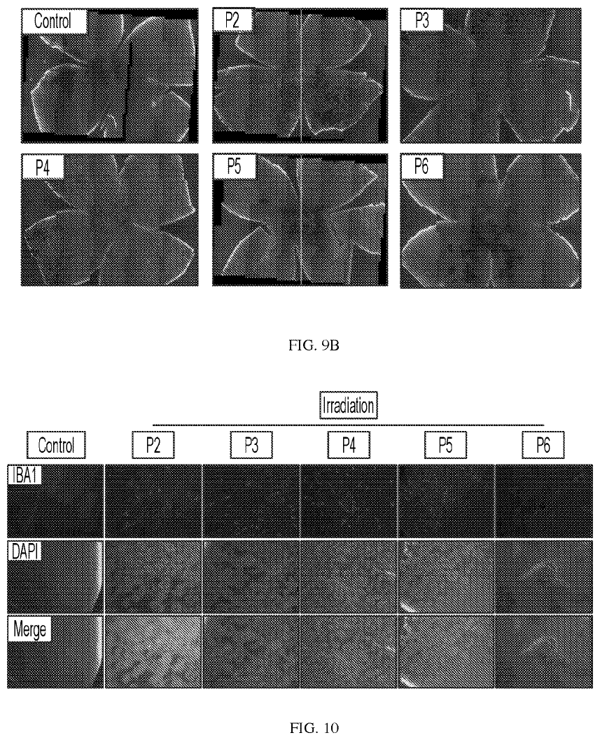

IR-induced microglia activation in mice with different genetic background to sec whether genetic background will influence the phenotypical changes in IR-RD mouse model were further examined. To do so, IR-RD in S129 wild type mice (FIGS. 9-11) were generated.

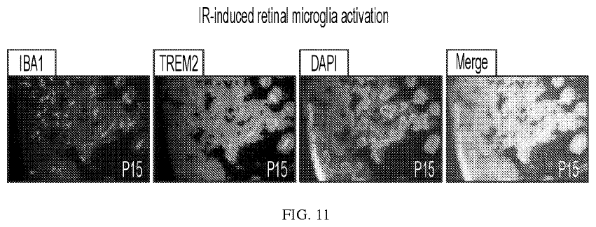

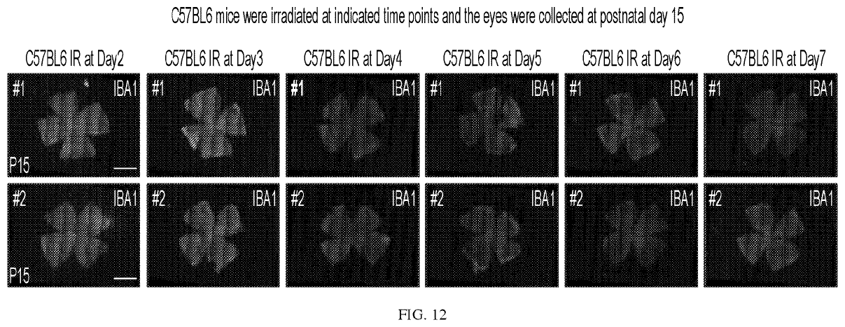

In FIGS. 9A-9B, S129 mice were irradiated at postnatal day (P) 2, 3, 4, 5 or 6 at the dose of 2.4 G and the eyes were examined at postnatal day 15 (P15). Representative flat mount images stained with DAPI for nuclei showed that “holes” in the areas surrounding active microglia as described in FIG. 2C were much more in the mice with IR at P2, P3 and P4, and reduced in the mice with IR at P5 and P5. These findings suggest that the time points of postnatal day 2 to 4 can induce more microglia compared to late time points at S129 mice. Enlarged images were showed in FIG. 10. In FIG. 11, Trem2 expression was examined in S129 mice.

### Example 7: IR-RD Model was Examined in Mice With Different Genetic Background—C57BL6 Mice

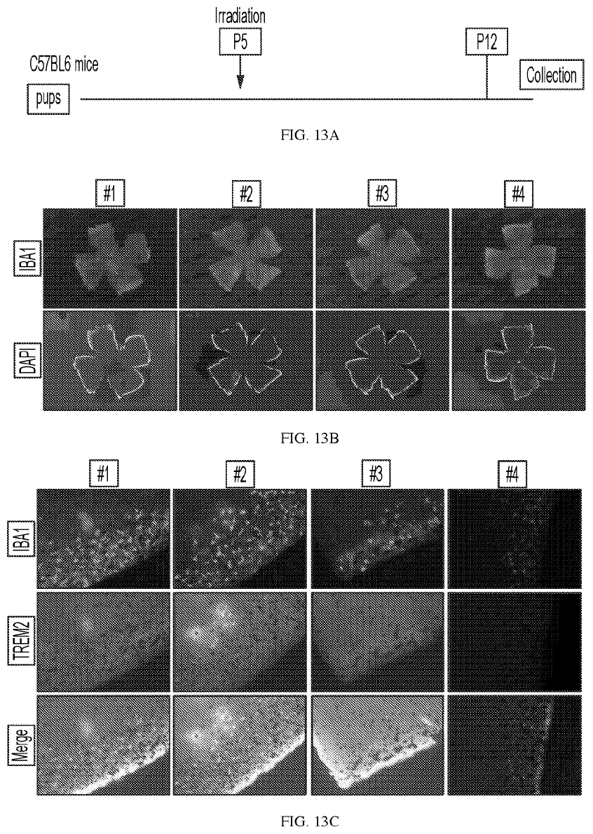

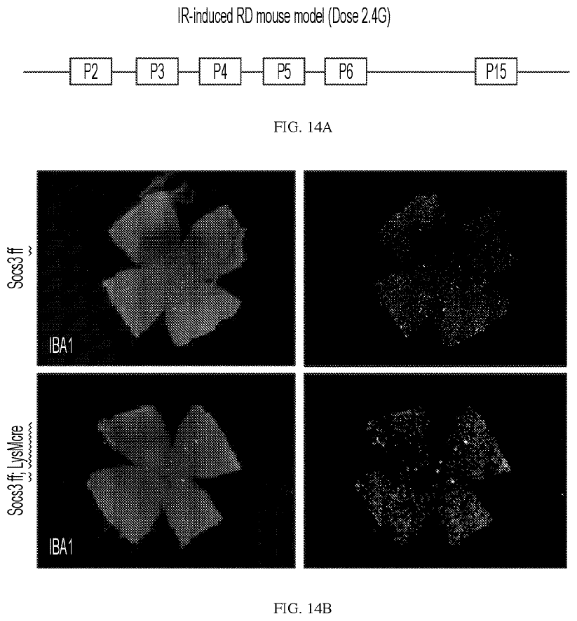

The IR-induced microglia activation in mice with different genetic background to see whether genetic background will influence the phenotypical changes in IR-RD mouse model were examined. The IR-RD in C57BL6 wild type mice (FIGS. 12-13C) were generated.

In FIG. 12, C57BL6 mice were irradiated at indicated time points and the population of microglia on flat mounts were examined at postnatal 15 by staining with active microglia marker IBA1. Microglia can be induced by IR at postnatal day 2, 3, 4, and 5, but very small number of IBA1+ cells on the flat mounts from mice with IR at postnatal 6 and 7.

In FIGS. 13A-13C, C57 BL6 mice were irradiated at the latest time point, postnatal P5 and the microglia were examined, and the images with high magnification were taken to show the morphonology of active microglia induced by IR.

### Example 8: IR-RD Mouse Model was Examined in Mice With Different Genetic Background—Myeloid SOCS3 Knockout Mice

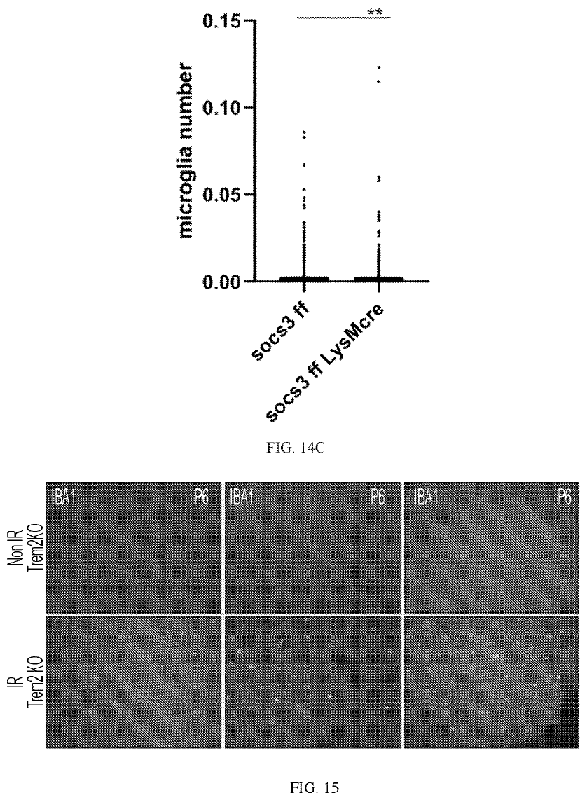

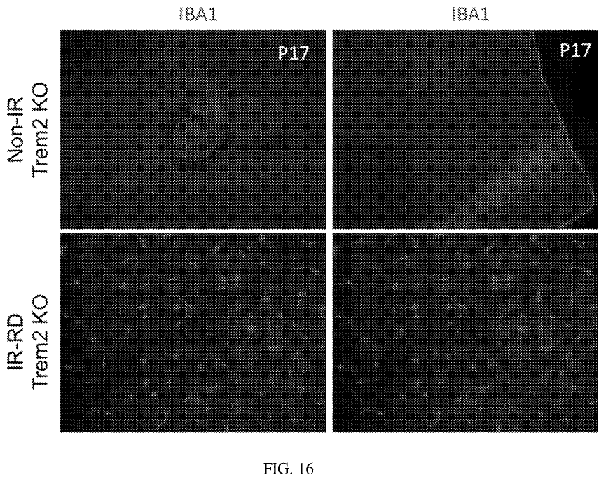

Neuronal and glial suppressor of cytokine signaling 3 (SOCS3) inhibits pathological retinal angiogenesis in OIR mice by suppressing vascular endothelial growth factor A (VEGFA) (10), and suppression of SOCS3 abolishes the anti-inflammatory and vaso-protective effects of retinoic-acid-receptor-related orphan receptor alpha deficiency in OIR mice (11). SOCS3 can regulate tissue inflammation and cytokine secretion (12-14) as well as control pathological ocular angiogenesis (10, 11, 15, 16). Additionally, myeloid cells accumulate in the areas of laser-induced choroidal NV (CNV) in myeloid Socs3 cKO mice (17). Therefore, the IR-induced microglia activation in myeloid SOCS3 deficient mice were investigated to determine whether myeloid SOCS3 can regulated IR-induced microglia (FIGS. 14A-14C).

In FIGS. 14A-14C, littermate myeloid SOCS3 knockout mice (Socs3ff;LysMcre) and Socs3 floxed control mice (Socs3 ff) were irradiated at postnatal day 2 at the dose of 2.4 G and the eyes were examined at postnatal 15. The numbers of microglia (IBA+ cells) were quantified and compared. The data showed that the number of IBA+ cells were significantly increased in myeloid SOCS3 knockout mice compared to littermate control mice, which were consistent with the findings that myeloid SOCS3 controls microglia accumulation and activation in other mouse models.

### Example 9: IR-RD Model was Examined in Mice With Different Genetic Background—Trem2 Knockout Mice

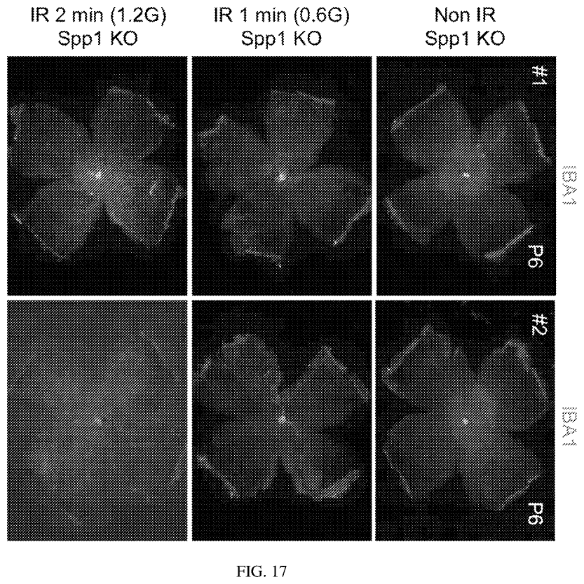

The IR-induced microglia activation in mice with different genetic background to see whether genetic background will influence the phenotypical changes in IR-RD mouse model were examined. IR-RD in Trem2 knockout mice (Trem2 KO) (FIG. 15-FIG. 16)) were generated. Trem2 expression was highly induced in IR-RD model. It was investigated whether microglia can be induced in Trem2KO mice, and the data showed that microglia was induced in Trem2 KO mice at both earlier (postnatal day 6 in FIG. 15) and late stages (postnatal 17 in FIG. 16).

### Example 10: IR-RD Model was Examined in Mice With Different Genetic Background—Trem2 Knockout Mice

The IR-induced microglia activation in mice with different genetic background to see whether genetic background will influence the phenotypical changes in IR-RD mouse model were examined. The IR-RD in Trem2 knockout (Trem2 KO) mice (FIG. 16) were generated.

### Example 11: IR-RD Model was Examined in Mice With Different Genetic Background—Spp1 Knockout Mice

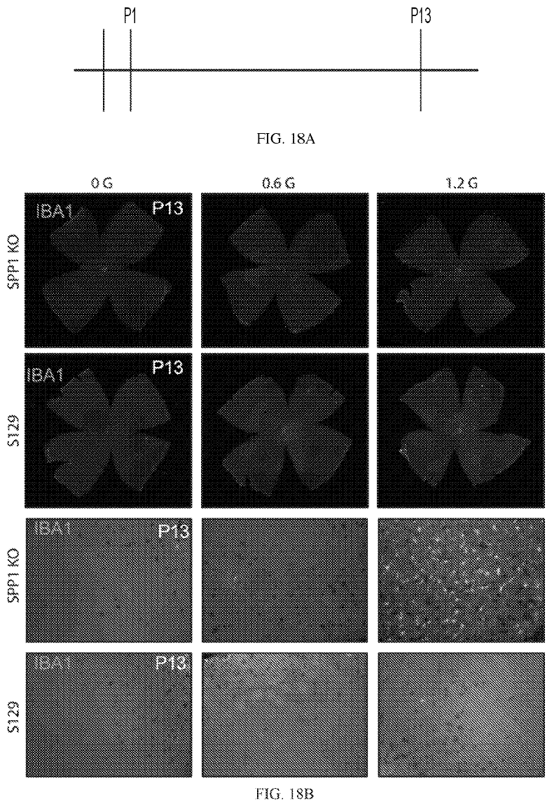

The IR-induced microglia activation in mice with different genetic background to see whether genetic background will influence the phenotypical changes in IR-RD mouse model were examined. The IR-RD in Spp1 knockout mice (Spp1 KO) (FIGS. 17-18B) were generated. Spp1 expression was highly induced in IR-RD model. It was examined whether microglia can be induced in Spp1 KO mice and the data showed that microglia was induced in Spp1 KO mice at both earlier (postnatal day 6 in FIG. 17) and late stages (postnatal 13 in FIGS. 18A-18B).

### Example 12: IR-RD Model was Examined in Mice With Different Genetic Background—Ai9 tdTomato Reporter Mice

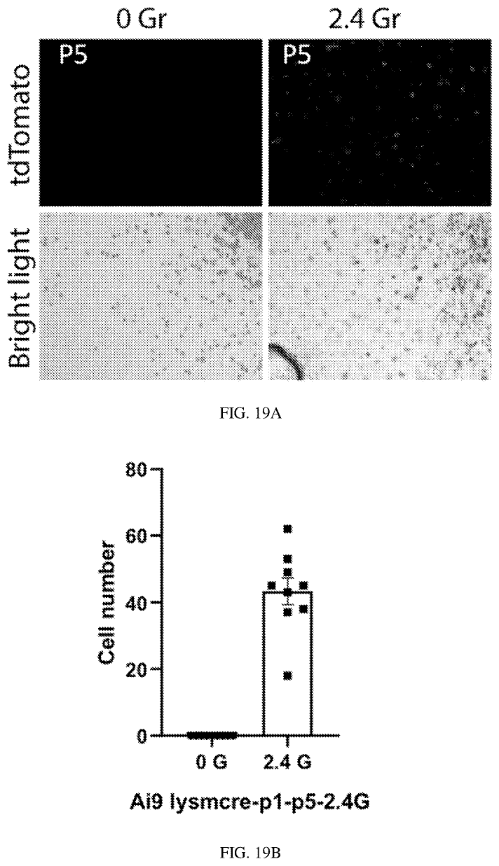

IR-induced microglia activation in mice with different genetic background to see whether genetic background will influence the phenotypical changes in IR-RD mouse model were examined. IR-RD in Ai9 tdTomato reporter mice (FIGS. 19A-19B) were generated. Myeloid specific CreLysM-driven Ai9 tandem dimer Tomato (tdTomato) reporter mice (Ai9 lysmcre) is a useful tool to study the microglia morphology and location during the disease progression. In this mouse strain, all the microglia will be labelled with tdTomato reporter. It was examined whether IR can induce microglia activation in this reporter mouse strain and the data showed that microglia can be successfully induced Ai9 reporter mice.

### Rod Photoreceptor SOCS3 Deficient Mice

### REFERENCES

1 Han, W., Umekawa, T., Zhou, K., Zhang, X. M., Ohshima, M., Dominguez, C. A., Harris, R. A., Zhu, C. and Blomgren, K. (2016) Cranial irradiation induces transient microglia accumulation, followed by long-lasting inflammation and loss of microglia. Oncotarget, 7, 82305-82323.
2 Greene-Schloesser, D. and Robbins, M. E. (2012) Radiation-induced cognitive impairment—from bench to bedside. Neuro Oncol, 14 Suppl 4, iv37-44.
3 Xu, H., Chen, M. and Forrester, J. V. (2009) Para-inflammation in the aging retina. Progress in retinal and eye research, 28, 348-368.
4 Wang, T., Zhou, P., Xie, X., Tomita, Y., Cho, S., Tsirukis, D., Lam, E., Luo, H. R. and Sun, Y. (2021) Myeloid lineage contributes to pathological choroidal neovascularization formation via SOCS3. EBioMedicine, 73, 103632.
5 Ulland, T. K. and Colonna, M. (2018) TREM2—a key player in microglial biology and Alzheimer disease. Nat Rev Neurol, 14, 667-675.
6 Wu, Y., Wang, M., Yin, H., Ming, S., Li, X., Jiang, G., Liu, Y., Wang, P., Zhou, G., Liu, L. et al. (2021) TREM-2 is a sensor and activator of T cell response in SARS-CoV-2 infection. Sci Adv, 7, eabi6802.
7 Wu, Y., Wu, M., Ming, S., Zhan, X., Hu, S., Li, X., Yin, H., Cao, C., Liu, J., Li, J. et al. (2021) TREM-2 promotes Th1 responses by interacting with the CD3zeta-ZAP70 complex following Mycobacterium tuberculosis infection. The Journal of clinical investigation, 131.
8 Coelho, I., Duarte, N., Barros, A., Macedo, M. P. and Penha-Goncalves, C. (2020) Trem-2 Promotes Emergence of Restorative Macrophages and Endothelial Cells During Recovery From Hepatic Tissue Damage. Frontiers in immunology, 11, 616044.
9 Sun, Y., Ju, M., Lin, Z., Fredrick, T. W., Evans, L. P., Tian, K. T., Saba, N. J., Morss, P. C., Pu, W. T., Chen, J. et al. (2015) SOCS3 in retinal neurons and glial cells suppresses VEGF signaling to prevent pathological neovascular growth. Science signaling, 8, ra94.
10 Wang, Z., Liu, C. H., Sun, Y., Gong, Y., Favazza, T. L., Morss, P. C., Saba, N. J., Fredrick, T. W., He, X., Akula, J. D. et al. (2016) Pharmacologic Activation of Wnt Signaling by Lithium Normalizes Retinal Vasculature in a Murine Model of Familial Exudative Vitreoretinopathy. The American journal of pathology, 186, 2588-2600.
11 Sun, Y., Lin, Z., Liu, C. H., Gong, Y., Liegl, R., Fredrick, T. W., Meng, S. S., Burnim, S. B., Wang, Z., Akula, J. D. et al. (2017) Inflammatory signals from photoreceptor modulate pathological retinal angiogenesis via c-Fos. J Exp Med, 214, 1753-1767.
12 Hood, D. C. and Birch, D. G. (1994) Rod phototransduction in retinitis pigmentosa: estimation and interpretation of parameters derived from the rod a-wave. Investigative ophthalmology & visual science, 35, 2948-2961.
13 Gong, Y., Li, J., Sun, Y., Fu, Z., Liu, C. H., Evans, L., Tian, K., Saba, N., Fredrick, T., Morss, P. et al. (2015) Optimization of an Image-Guided Laser-Induced Choroidal Neovascularization Model in Mice. PloS one, 10, e0132643.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one member of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. An irradiated mouse which has been exposed to a dose of ionizing radiation, wherein the mouse comprises one or more retina exhibiting microglial activation, neovascularization, and/or photoreceptor degeneration.

2. The irradiated mouse of claim 1, wherein the mouse has a wild-type (WT) genetic background.

3. The irradiated mouse of claim 1, wherein the mouse has an Ai9 f/f/::LysMCre, Ai9f/f:: Cx3cr1 Cre, Ai9f/f:: Tmem119 Cre, Ai9f/f:: P2ry12 Cre, Cx3cr1-GFP, or mTmG::Cx3cr1Cre genetic background.

4. The irradiated mouse of any one of claims 1-3, wherein the mouse comprises an increased number of microglial cells in the subretinal space, as compared to the number of microglial cells in the subretinal space before exposure to the dose of ionizing radiation.

5. The irradiated mouse of any one of claims 1-4, wherein the mouse comprises increased growth of retinal blood vessels, as compared to the growth of retinal blood vessels before exposure to the dose of ionizing radiation.

6. The irradiated mouse of any one of claims 1-5, wherein the mouse comprises an increased level of cell death in the photoreceptor layer of the retina, as compared to the level of cell death in the photoreceptor layer of the retina before exposure to the dose of ionizing radiation.

7. The irradiated mouse of any one of claims 1-6, wherein the mouse comprises a retina of reduced thickness, as compared to the thickness of the retina before exposure to the dose of ionizing radiation.

8. The irradiated mouse of claim 7, wherein the mouse comprises a retina comprising a photoreceptor layer of reduced thickness, as compared to the thickness of the photoreceptor layer before exposure to the dose of ionizing radiation.

9. The irradiated mouse of any one of claims 1-8, wherein the dose of ionizing radiation comprises beta radiation and/or gamma irradiation.

10. The irradiated mouse of any one of claims 1-9, wherein the dose of ionizing radiation comprises at least 1.2 Gy of ionizing radiation.

11. The irradiated mouse of claim 10, wherein the dose of ionizing radiation comprises 2.4 Gy of ionizing radiation.

12. The mouse of any one of claims 1-11, wherein has been exposed to the dose of ionizing radiation within 10 days of birth.

13. The mouse of any one of claims 1 and 3-12, wherein the mouse comprises an inactivated gene that is involved or suspected to be involved in microglial activation, neovascularization, and/or photoreceptor degeneration of the retina.

14. The mouse of claim 13, wherein the inactivated gene is TREM2 and/or SPP1.

15. A method for producing a mouse model of ionizing radiation-induced retinal degeneration, the method comprising administering to a mouse a dose of ionizing radiation, wherein the dose of ionizing radiation is sufficient to induce microglial activation, neovascularization, and/or photoreceptor degeneration in one or more retina of the mouse.

16. The method of claim 15, wherein the mouse has a wild-type (WT) genetic background.

17. The method of claim 15, wherein the mouse has an Ai9 f/f/::LysMCre genetic background.

18. The method of any one of claims 15-17, wherein the administration results in an increased number of microglial cells in the subretinal space of the mouse, as compared to the number of microglial cells in the subretinal space of the mouse before the administration.

19. The method of any one of claims 15-19, wherein the administration results in increased growth of retinal blood vessels, as compared to the growth of retinal blood vessels before the administration.

20. The method of any one of claims 15-19, wherein the administration results in an increased level of cell death in the photoreceptor layer of the retina of the mouse, as compared to the level of cell death in the photoreceptor layer of the retina of the mouse before the administration.

21. The method of any one of claims 15-20, wherein the administration results in a decrease in the retinal thickness of the mouse, as compared to the retinal thickness of the mouse before the administration.

22. The method of claim 21, wherein the administration results in a decrease in the photoreceptor layer thickness of the mouse, as compared to the photoreceptor layer thickness of the mouse before the administration.

23. The method of any one of claims 15-22, wherein the administration comprises beta radiation and/or gamma irradiation.

24. The method of claim 23, wherein the administration comprises ionizing radiation from a Cesium-137 (¹³⁷Cs) radioactive source.

25. The method of any one of claims 15-24, wherein the administration comprises at least 1.2 Gy of ionizing radiation.

26. The method of any one of claims 15-25, wherein the administration comprises 2.4 Gy of ionizing radiation.

27. The method of any one of claims 15-26, wherein the administration occurs within 10 days of birth of the mouse.

28. The method of any one of claims 15 and 17-27, wherein the mouse comprises an inactivated gene that is suspected to be involved in microglial activation, neovascularization, and/or photoreceptor degeneration of the retina.

29. The method of claim 28, further comprising assessing whether the gene is involved in microglial activation, neovascularization, and/or photoreceptor degeneration of the retina.

30. A method comprising administering to the mouse of any one of claims 1-14 a therapeutic agent for the treatment of retinal degeneration.

31. The method of claim 30, further comprising assessing the effectiveness of the drug in treating retinal degeneration.
